(19) United States
(12) Patent Application Publication
(10) Pub. No.: US 2016/0035247 A1
(43) Pub. Date: Feb. 4, 2016

(54) VISUAL FEEDBACK GENERATION IN TRACING A PATTERN

(71) Applicant: Ohio University, Athens, OH (US)

(72) Inventors: Chang Liu, Athens, OH (US); Siang Lee Hong, Canal Winchester, OH (US)

(73) Assignee: OHIO UNIVERSITY, Athens, OH (US)

(21) Appl. No.: 14/812,721

(22) Filed: Jul. 29, 2015

Related U.S. Application Data
(60) Provisional application No. 62/030,311, filed on Jul. 29, 2014.

(51) Int. Cl. G09B 19/00; G09B 7/00; A61B 5/11

(52) U.S. Cl. CPC G09B 19/003; A61B 5/1124; G09B 7/00

(57) ABSTRACT

Systems and methods include generating visual feedback from a traced pattern that is completed by an individual using a multi-touch device. Embodiments of the present disclosure relate to displaying a previously generated pattern by a user interface of the multi-touch device to the individual for the individual to trace. The traced pattern generated from continuously tracing the previously generated pattern by the individual from an initial point on the previously generated pattern to an end point on the previously generated pattern is received via the user interface of the multi-touch device. Visual feedback is generated via the user interface that translates motion-based behavior data associated with the traced pattern to a visible depiction of the motion-based behavior data.

### CROSS-REFERENCE TO RELATED APPLICATIONS

This application is a U.S. Nonprovisional Application which claims the benefit of U.S. Provisional Application No. 62/030,311 filed on Jul. 29, 2014, which is incorporated herein by reference in its entirety.

### BACKGROUND

As individuals age and/or are debilitated by degenerative disorders such as Parkinson's disease, the individuals may experience a decrease in their cognitive abilities to maintain velocity and/or precision in their movements. Such a decline in movement control may have a significant impact on the individuals' fine movements which may hinder impacted individuals to execute everyday tasks that require fine movements such as buttoning a shirt and/or operating a vehicle. As the difficulty in executing everyday tasks increases for impacted individuals, the independence and self-sufficiency of the impacted individuals decrease.

Typically, as individuals experience a decrease in their cognitive abilities to maintain the precision in their movements, the impacted individuals compensate for their decrease in precision by executing their movements with slower velocities. For example, an individual that has experienced a decrease in their cognitive abilities to maintain the precision in their movements, moves a glass of water across a table at a slower velocity to compensate for their decrease in precision so that they have a higher rate of success in preventing the water from spilling out of the glass. However, such a decrease in the velocity in the movements of impacted individuals' movements in completing everyday tasks does not slow the rate in which the movement control of the impacted individuals continues to decline. Rather, impacted individuals should continually push themselves to execute tasks at higher velocities and/or engage in therapeutic exercises that sharpen the precision of the impacted individual in executing movements to slow the rate at which their movement control declines.

Conventionally, individuals that are impacted with a significant decrease in their cognitive abilities to maintain the precision in their movements are diagnosed as such by physicians and are then referred to physical and/or occupational therapists. Through a series of sessions, the therapists may engage the impacted individuals with various exercises and/or treatments that may slow the rate at which the impacted individuals' movement control continues to decline. However, the series of sessions that the impacted individuals engage the therapists are typically limited in quantity and time. For example, an impacted individual may be referred to a physical therapist to engage the physical therapist for two sessions a week where each session is limited to an hour for a total quantity of thirty sessions.

As a result, the impacted individual is limited to engaging exercises and/or treatments to slow the rate of decline in their movement control to two hours a week for fifteen weeks. Impacted individuals that fail to be diagnosed by a physician as having a significant decline in their movement control may not even be referred to a therapist. Further, individuals that may not yet have a significant decline in their movement control to be diagnosed as such and referred to a therapist may not have the wherewithal to engage in convenient exercises on their own to prevent a significant decline in their movement control.

### BRIEF SUMMARY

Embodiments of the present invention relate to generating visual feedback to an individual that is executing a trace of a previously generated pattern, such as a pattern generated by another individual, via a multi-touch device so that the individual may understand how the individual's trace compares to the previously generated pattern. In an embodiment, a method generates visual feedback from a comparison of a traced pattern that is completed by an individual using a multi-touch device. The previously generated pattern may be displayed by a user interface of the multi-touch device to the individual for the individual to trace. The traced pattern generated from continuously tracing the previously generated pattern by the individual from an initial point on the previously generated pattern to an end point on the previously generated pattern via the user interface of the multi-touch device may be received. Visual feedback may be generated via the user interface that translates motion-based behavior data associated with the traced pattern to a visible depiction of the motion-based behavior data.

In an embodiment, a multi-touch device generates visual feedback from a traced pattern that is completed by an individual. A user interface is configured to display the previously generated pattern by a user interface of the multi-touch device to the individual for the individual to trace. A transceiver is configured to receive the traced pattern generated from continuously tracing the previously generated pattern by the individual from an initial point on the previously generated pattern to an end point on the previously generated pattern via the user interface. A generating module is configured to generate visual feedback via the user interface that translates motion-based behavior data associated with the traced pattern compares to a visible depiction of the motion-based behavior data associated with the previously generated pattern.

Further embodiments, features, and advantages, as well as the structure and operation of the various embodiments, are described in detail below with reference to the accompanying drawings.

### BRIEF DESCRIPTION OF THE DRAWINGS/FIGURES

Embodiments are described with reference to the accompanying drawings. In the drawings, like reference numbers may indicate identical or functionally similar elements.

FIG. 1 shows an illustration of visual feedback system;

FIG. 2 is a flowchart showing an example method of generating visual feedback from a traced pattern that is completed by the individual;

FIG. 3 depicts an example visual feedback configuration that is translating the motion-based behavior skill of velocity into visual feedback; and

FIG. 4 depicts a detailed view of an exemplary visual feedback system for generating visual feedback from a traced pattern that is completed by the individual.

### DETAILED DESCRIPTION

In the Detailed Description herein, references to “one embodiment”, “an embodiment”, an “example embodiment”, etc., indicate that the embodiment described may include a particular feature, structure, or characteristic, by every embodiment may not necessarily include the particular feature, structure, or characteristic. Moreover, such phrases are not necessarily referring to the same embodiment. Further, when a particular feature, structure, or characteristic may be described in connection with an embodiment, it may be submitted that it may be within the knowledge of one skilled in the art to affect such feature, structure, or characteristic in connection with other embodiments whether or not explicitly described.

The following detailed description refers to the accompanying drawings that illustrate exemplary embodiments. Other embodiments are possible, and modifications can be made to the embodiments within the spirit and scope of this description. Those skilled in the art with access to the teachings provided herein will recognize additional modifications, applications, and embodiments within the scope thereof and additional fields in which embodiments would be of significant utility. Therefore, the detailed description is not meant to limit the embodiments described below.

In an embodiment, an individual that is experiencing a decline in their movement control may slow the rate at which their movement control continues to decline by tracing patterns. In executing a trace of a pattern, the individual implements cognitive skills in maintaining the precision necessary to trace the pattern without going outside the boundaries of the pattern. However, simply tracing the pattern with precision may not be sufficient to slow the rate of decline of the individual's movement control. The individual may compensate for their decline in movement control to maintain their precision in tracing the pattern by allowing other motion-based behavior skills to lapse. Such a lapse in other motion-based behavior skills to improve the precision in tracing the pattern may not be sufficient to slow the rate of decline of the individual's movement control.

For example, the individual in maintaining the motion-based behavior skill of precision in tracing the pattern significantly decreases the velocity in which the individual traces the pattern. Allowing the motion-based behavior skill of velocity to lapse to maintain the precision in tracing the pattern may have a significantly less impact if any in slowing the rate of decline in the movement control of the individual. The individual may experience a much greater impact in slowing the rate of decline in their movement control when the individual maintains each motion-based behavior skill at an adequate level when completing the trace of the pattern. For example, the individual may experience a much greater impact in slowing the rate of decline in the movement control when the individual completes the trace with precision without decreasing the velocity in completing the trace to maintain the precision.

The individual may experience a further impact in slowing the rate of decline in their movement when the level of each motion-based behavior skill that the individual is to strive for when completing the trace of the pattern is determined by another individual. If the individual has experienced a significant decline in their movement control, the individual may no longer have the aptitude to independently grasp the level that each motion-based behavior skill is to reach when completing the trace of the pattern to adequately slow the rate of decline in their movement control. As a result, another individual that has experienced minimal decline in their movement control may first complete a pattern with motion-based behavior skills at higher levels than the individual that suffering a decline in their movement control. The individual may then attempt to trace the pattern completed by the other individual and in doing so strive to have each motion-based behavior skill reach the level that the other individual reached for each respective motion-based behavior skill.

In order to maintain each motion-based behavior skill at an adequate level when tracing the pattern, the individual may require some type of feedback when completing the trace to understand the status of each motion-based behavior skill. The individual may easily receive feedback on the precision of the trace based on the portions of the trace that are within the boundaries of the pattern versus the portions of the trace that are outside the boundaries of the pattern. However, the individual may not easily receive feedback from other motion-based behavior skills when completing the trace of the pattern. For example, the individual may not easily gauge the velocity that the individual is completing each portion of the trace with.

Translating each motion-based behavior skill implemented by the individual when completing the trace to visual feedback may improve the individual's ability to maintain each motion-based behavior skill at an adequate level to slow the rate of decline in the movement of the individual. For example, translating the velocity that the individual completes each portion of the trace with to visual feedback may enable the individual to easily recognize that the individual needs to increase the velocity when completing the trace.

The generation of visual feedback to the individual that visually depicts the performance of motion-based behavior skills that otherwise would be difficult for the individual to engage may increase the effectiveness in the individual to maintain such skills to slow the decline in their movement control. The convenience in tracing a pattern coupled with the visual feedback generated when tracing the pattern enables the individual to continue to perform exercises that may slow the decline in their movement control outside of therapeutic sessions with a therapist. Also, the generation of a pattern by another individual that has not experienced a significant decline in their movement control may provide a clear benchmark for the individual to strive for when completing each trace of the pattern previously traced by the other individual.

System Overview

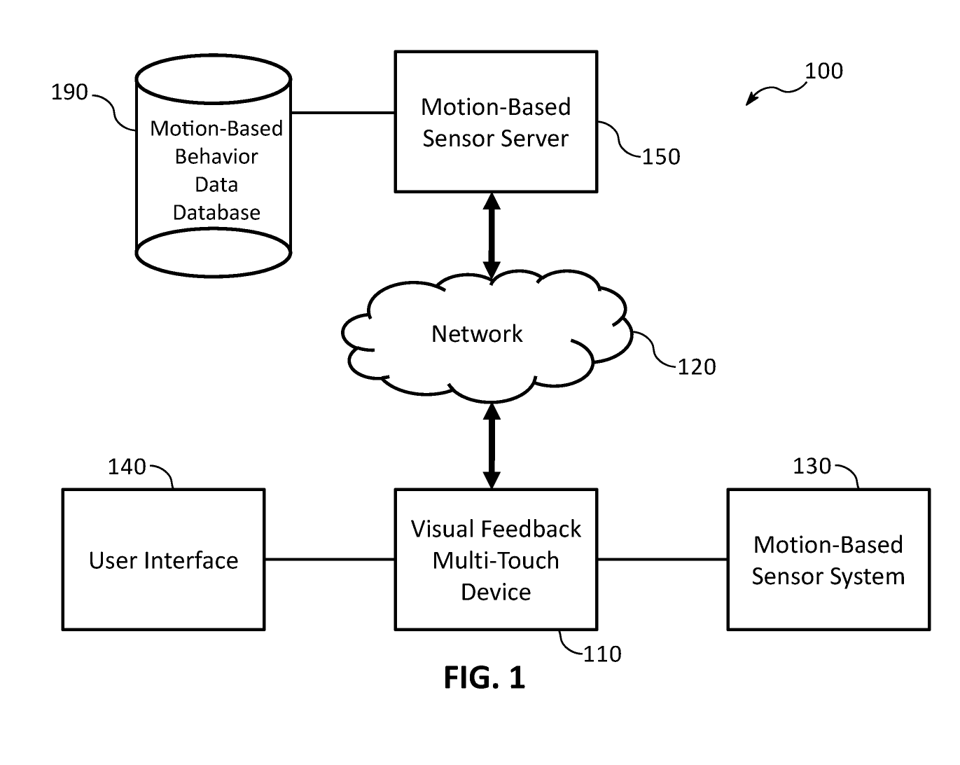

As shown in FIG. 1, visual feedback generation system 100 includes a visual feedback multi-touch device 110, a network 120, a motion-based sensor system 130, a user interface 140, a motion-based sensor server 150, and a motion-based behavior database 190.

Visual feedback multi-touch device 110 may be a device that is capable of electronically communicating with other devices while having a multi-touch display. The multi-touch display has the ability to recognize the presence of two or more points in contact with the surface of the multi-touch display. Examples of visual feedback multi-touch device 110 may include a mobile telephone, a smartphone, a workstation, a portable computing device, other computing devices such as a laptop, or a desktop computer, cluster of computers, set-top box, a computer peripheral such as a printer, a portable audio, and/or video player, a payment system, a ticketing writing system such as a parking ticketing system, a bus ticketing system, a train ticketing system or an entrance ticketing system to provide some examples, or in a ticket reading system, a toy, a game, a poster, packaging, an advertising material, a product inventory checking system and or any other suitable electronic device with a multi-touch display that will be apparent to those skilled in the relevant art(s) without departing from the spirit and scope of the invention.

In an embodiment, multiple modules may be implemented on the same multi-touch device. Such a multi-touch device may include software, firmware, hardware, or a combination thereof. Software may include one or more applications on an operating system. Hardware can include, but is not limited to, a processor, memory, and/or graphical user interface display. Visual feedback multi-touch device 110 may store the motion-based behavior data captured by motion-based sensor system 130.

An individual engaged in an identity authentication session may interact with visual feedback multi-touch device 110 via user interface 140. User interface 140 may include a multi-touch display that has the ability to recognize the presence of two or more points in contact with the surface of the multi-touch display. User interface 140 may include any type of display device including but not limited to a touch screen display, a cathode ray tube (CRT) monitor, a liquid crystal display (LCD) screen, and/or any other type of display device that includes a multi-touch display that will be apparent from those skilled in the relevant art(s) without departing from the spirit and scope of the present disclosure.

One or more motion-based sensor systems 130 may connect to one or more visual feedback multi-touch devices 110. Motion-based sensor system 130 may include one or more sensors that capture motion-based data that is the physical movement of an individual. Motion-based sensor system 130 may include a video imaging system, an infrared imaging system, a photographic imaging system, an air sensing system, a thermal sensing system, a motion sensor that is capable of capturing two-dimensional data with a commercially available device such as a Kinect motion sensing input device by Microsoft, other motion sensing systems that include sensors that are associated with a multi-touch communications device that that can also be used without departing from the spirit and scope of the present disclosure. Motion-based sensor system 130 detects motion-based behavior data as the individual executes a series of motions when continuously touching the multi-touch display of user interface 140. For example, motion-based sensor system 130 can detect a sequence of positions the individual follows on the multi-touch display of user interface 140 when tracing a pattern displayed by user interface 140. Motion-based sensor system 130 tracks the velocity of the individual's movements over time as the individual traces the pattern as well as other variables, such as location relative to the pattern, as is explained hereinafter.

As shown, visual feedback multi-touch device 110 streams the motion-based behavior data to motion-based sensor server 150 via network 120. Network 120 includes one or more networks, such as the Internet. In some embodiments of the present invention, network 120 may include one or more wide area networks (WAN) or local area networks (LAN). Network 120 may utilize one or more network technologies such as Ethernet, Fast Ethernet, Gigabit Ethernet, virtual private network (VPN), remote VPN access, a variant of IEEE 802.11 standard such as Wi-Fi, and the like. Communication over network 120 takes place using one or more network communication protocols including reliable streaming protocols such as transmission control protocol (TCP). These examples are illustrative and not intended to limit the present invention.

One or more motion-based sensor servers 150 may connect to one or more visual feedback multi-touch devices 110 via network 120. Motion-based sensor servers 150 may include a data acquisition system, a data management system, intranet, conventional web-server, e-mail server, or file transfer server modified according to one embodiment. Motion-based sensor server 150 is typically a device that includes a processor, a memory, and a network interface, hereinafter referred to as a computing device or simply “computer.” Motion-based sensor server 150 may store the motion-based behavior data captured by motion-based sensor system 130.

Visual feedback multi-touch device 110, motion-based sensor server 150, and motion-based behavior data database 190 may share resources via network 120. For example, motion-based sensor server 150 may retrieve previously captured motion-based behavior data from the motions generated by the individual during previous pattern tracing sessions via network 120. Visual feedback multi-touch device 110 may also provide motion-based behavior data captured from the individual when tracing the pattern during each identity authentication session via network 120. Based on the cloud computing configuration, the interaction between visual feedback multi-touch device 110, motion-based sensor server 150, and motion-based behavior data database 190 may not be limited to a single computing device. For example, a plurality of computing devices may update motion-based behavior data database 190 via network 120 with captured motion-based behavior data.

### Visual Feedback Generation

Visual feedback multi-touch device 110 may generate visual feedback regarding the motion-based behavior data captured by visual feedback multi-touch device 110 as the individual traces a pattern. The pattern that the individual traces may be a previously generated pattern generated with previously captured motion-based behavior data previously captured by visual feedback multi-touch device 110. An embodiment consistent with the invention generates visual feedback to the individual that translates each motion-based behavior data generated from the individual's trace of the previously generated pattern to a visible depiction of the motion-based behavior data. The visual depiction of the motion-based behavior data may improve the individual's comprehension of the status of the motion-based behavior data when completing the trace of the previously generated pattern. Such an improved comprehension may enable the individual to improve upon particular motion-based behavior skills if necessary to slow the rate of decline in the motion control of the individual.

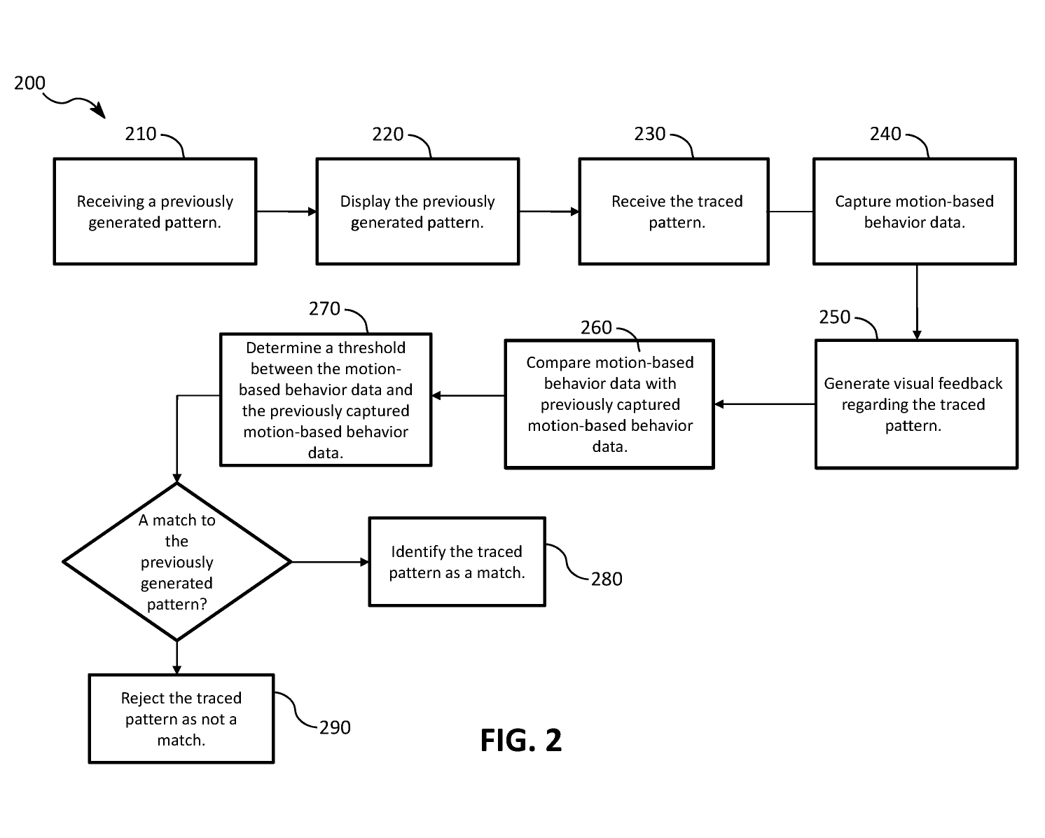

One such implementation of generating visual feedback from a traced pattern that is completed by the individual is illustrated by process 200 in FIG. 2. Process 200 includes nine primary steps: receive a previously generated pattern 210, display the previously generated pattern 220, receive the traced pattern 230, capture motion-based behavior data 240, generate visual feedback regarding the traced pattern 250, compare motion-based behavior data with previously captured motion-based behavior data 260, determine a threshold between the motion-based behavior data and the previously captured motion-based behavior data 270, identify the traced pattern as a match 280, and reject the traced pattern as not a match 290. Steps 210-290 are typically implemented in a computer, e.g., via software and/or hardware, e.g., visual feedback multi-touch device 110 of FIG. 1.

In step 210, a previously generated pattern may be received by visual feedback multi-touch device 110. The previously generated pattern may be a pattern that includes a series of points and/or continuous paths. As will be discussed in further detail below, the previously generated pattern is to be traced by an individual. Thus, the previously generated pattern may be received by visual feedback multi-touch device 110 before receiving the trace of the previously generated pattern from the individual. The individual may be any person that is engaging visual feedback multi-touch device 110 with the intent to trace the previously generated pattern to receive visual feedback regarding the motion-based behavior data generated when the individual completes the trace of the previously generated pattern. Motion-based behavior data will be discussed in further detail below.

The previously generated pattern may be generated by another individual and/or computing device independent and separate from the individual mentioned above that is engaging the visual feedback multi-touch device 110 with the intent to trace the previously generated pattern to receive visual feedback. The previously generated pattern may also be generated by the individual. For example, the previously generated pattern is generated by a physical therapist that is treating the individual to slow the rate in the decline of the individual's movement control. In another example, the previously generated pattern is generated by another individual that has not experienced a significant decline in their movement control.

Movement control is the ability of a human to control voluntary movements.

Particularly, movement control includes fine motor skills that provide a human the ability to coordinate small muscle movements which occur in body parts of the human in coordination with the eyes of the human. The previously generated pattern may be generated by initiating the previously generated pattern with an initial point on user interface 140 and then continuously creating the previously generated pattern without terminating contact with user interface 140 until an end point on user interface 140 is reached thus completing the previously generated pattern. The previously generated pattern may be a two-dimension pattern that is generated via user interface 140 in two-dimensional space.

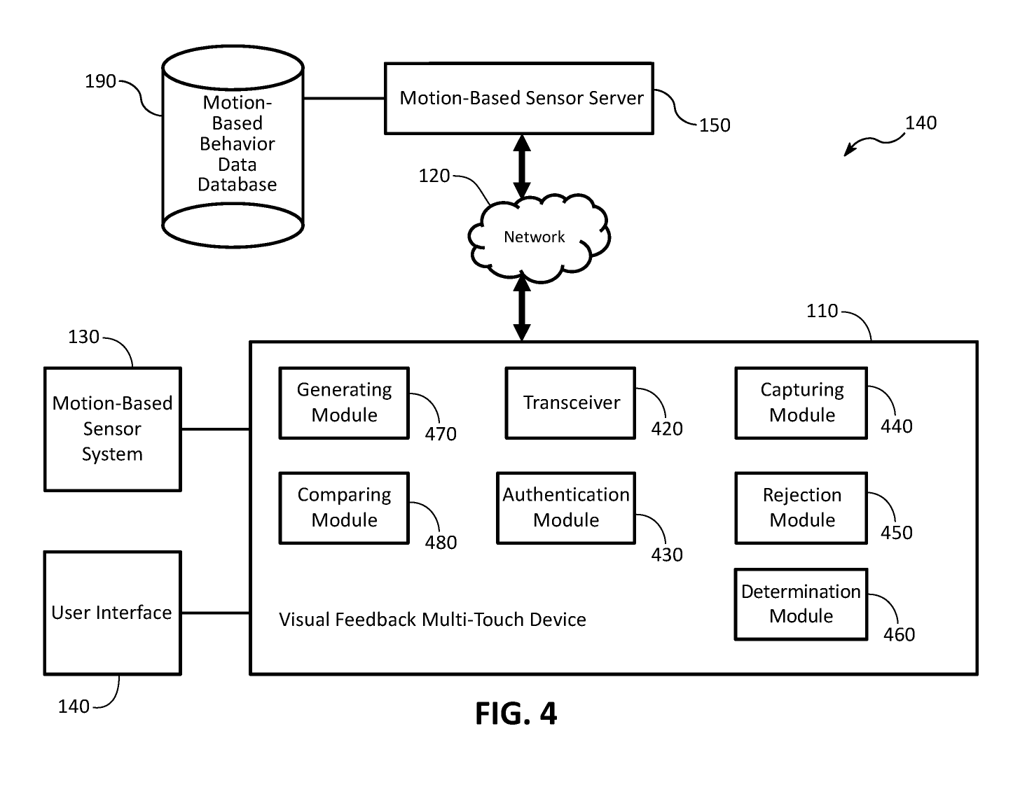

The previously generated pattern may be generated with previously captured motion-based behavior data that may be captured by motion-based sensor system 130. The previously generated pattern may be generated by another individual, computing device, the individual, and/or any other source capable of generating the previously generated pattern so that motion-based behavior data may be captured by motion-based sensor system 130 that will be apparent to those skilled in the relevant art(s) without departing from the spirit and scope of the present disclosure. In an example embodiment, step 210 may be performed by transceiver 420 as shown in FIG. 4 and discussed in more detail below.

After the previously generated pattern is received by visual feedback multi-touch device 110, in step 220, the previously generated pattern may be displayed via user interface 140. In an embodiment, user interface 140 may display the previously generated pattern for the individual to trace via the multi-touch display. In another embodiment, user interface 140 may also audibly announce to the individual the pattern included in the authentication template that the individual is to trace via the multi-touch display. The individual may be prompted with the pattern to trace with any other method that adequately identifies to the individual of the pattern that the individual is to trace that will be apparent to those skilled in the relevant art(s) without departing from the spirit and scope of the present disclosure. In an example embodiment, step 220 may be performed by user interface 140 as shown in FIG. 4 and discussed in more detail below.

After the previously generated pattern is displayed to the individual via user interface 140, in step 230, a traced pattern generated as the individual traces the previously generated pattern displayed by user interface 140 via the multi-touch display may be received. The traced pattern may be received as the individual executes the plurality of motions to continuously trace the pattern from an initial point to an end point via the multi-touch display of user interface 140. The individual decides to begin the trace of the pattern at an initial point on the pattern and then continues to trace the pattern by following a path along the pattern until the pattern is traced completing the pattern at an end point.

In an embodiment, the initial point and the end point may be at different locations on the pattern. In another embodiment, the initial point and the end point may be at substantially similar locations on the pattern where the individual begins and ends the trace in substantially similar locations on the pattern. The individual traces the pattern by continuously maintaining contact with the multi-touch display of user interface 140 from the initial point to the end point. The continuously traced pattern may be received via user interface 140 as the individual traces the pattern from the initial point to the end point. In an example embodiment, step 230 may be performed by transceiver 420 as shown in FIG. 4 and discussed in more detail below.

In step 240, motion-based behavior data that may be generated by the plurality of motions executed by the individual when continuously tracing the previously generated pattern may be captured. Motion capturing sensors included in motion-based sensor system 130 may capture the motion-based behavior data as the individual executes the plurality of motions when tracing the previously generated pattern. The motion-based behavior data includes data that is the result of motion-based behavior skills that the individual implements when tracing the previously generated pattern. The motion-based behavior skills include the skills implemented by the individual to coordinate muscle movements in coordination with the individual's eyes to complete the trace of the previously generated pattern. For example, the individual implements the motion-based behavior skill of precision in maintaining the trace within the outline of the previously generated pattern while the motion-based behavior data that results from the motion-based behavior skill of precision are the corresponding x-coordinates and y-coordinates of the trace.

The captured motion-based behavior data that when translated to visual feedback, which is to be discussed in greater detail below, may have an impact in slowing the decline in the individual's movement control. The individual may focus on maintaining the motion-based behavior skills associated with the captured motion-based behavior data at adequate levels so that the decline in the individual's movement control may be slowed. For example, the captured motion-based behavior data is the amount of time taken by the individual to complete the trace of the previously generated pattern as the individual begins the trace with the initial point and completes the trace with the end point. The amount of time taken by the individual to complete the trace is captured from the sensors coupled to the multi-touch display of user interface 140 included in motion-based sensor system 130.

The individual may then focus on the amount of time the individual takes to complete the trace to have an impact in slowing the decline in the individual's movement control. The individual may make a conscious effort to decrease the amount of time the individual takes to complete the trace while maintaining the precision in completing the trace. The individual may continually complete the trace repetitiously while decreasing the time to complete the trace and maintaining the precision each time the individual completes the trace. Such a focus on these motion-based behavior skills depicted by the captured motion-based behavior data related to these skills may have an impact on slowing the decline of the individual's movement control.

Motion-based sensor system 130 may be coupled to the multi-touch display of user interface 140 so that motion-based sensor system 130 may capture the motion-based behavior data generated as the individual engages the pattern by maintaining contact with the multi-touch display. The individual may also be within proximity of the multi-touch display so that the motion capturing sensors included in motion-based sensor system 130 that are coupled to the multi-touch display can adequately capture the motion-based behavior data generated from the plurality of motions executed by the individual when tracing the pattern via the multi-touch display. Motion-based sensor system 130 may continuously capture the motion-based behavior data beginning with the initial point of the individual's continuous trace through the end point of the individual's trace of the pattern. The plurality of motions executed by the individual that generate the motion-based behavior data may include any bodily motion and/or relation between bodily motions that occur as the individual traces the pattern.

The motion-based behavior data may include but is not limited to the initial point and end point selected by the individual to begin and complete the trace, the amount of time taken by the individual to complete the trace, the coordinates of the trace relative to the previously generated pattern, the velocities in completing the trace, the sequence of the points connected during the trace, the sequence of the continuous path that is followed during the trace, the pressure applied to the multi-touch display of user interface 140 by the individual as the individual completes the trace and/or any other motion-based behavior data that when focused on by the individual slows the decline in movement control of the individual that will be apparent to those skilled in the relevant art(s) without departing from the spirit and scope of the present disclosure. The above discussion relative to capturing the motion-based behavior data generated from the trace of the previously generated pattern by the individual also is applicable to the previously captured motion-based behavior data generated during the creation of the previously generated pattern received in step 210 and displayed in step 220. In an example embodiment, step 240 may be performed by capturing module 440 as shown in FIG. 4 and discussed in more detail below.

The captured motion-based behavior data and previously captured motion-based behavior data is stored in motion-based behavior data database 190. The captured motion-based behavior data and previously motion-based behavior data is stored in motion-based behavior data database 190 as associated with the individual and/or computing device that generated the motion-based behavior data. The captured motion-based behavior data and previously captured motion-based behavior data associated with the generator of such data as stored in motion-based behavior data database 190 may then be referenced in determining thresholds to be applied to a matching application that is to be discussed in further detail below.

In step 250, visual feedback may be generated regarding the trace of the previously generated pattern. The visual feedback may translate the motion-based behavior data associated with the trace to a visible depiction of the motion-based behavior data. The visible depiction of the motion-based behavior data may be sufficient so that the individual may comprehend the status of the motion-based behavior data as the individual completes the trace.

As noted above, the individual that is experiencing a decrease in their movement control may compensate for their lack of precision in tracing the previously generated pattern by slowing down other motion-based behavior skills, such as velocity. The individual may compensate the velocity for precision in which the individual traces the pattern with a slower velocity to more precisely trace the pattern within the boundaries of the pattern. However, slowing down the velocity in which the individual traces the pattern in order to improve precision may have a minimum impact if any on slowing the rate in which the individual's movement control degrades. In order for the individual to minimize the rate at which their movement control degrades, the individual may have to maintain other motion-based behavior skills other than precision while completing the trace of the previously generated pattern.

However, other motion-based behavior skills, such as velocity, may be difficult for the individual to comprehend without translating the motion-based behavior skills into visual feedback. Visual feedback visually depicts the motion-based behavior skills to the individual in a manner that the individual may easily comprehend the status of the motion-based behavior skills and make the efforts to adjust the status of the motion-based behavior skills accordingly.

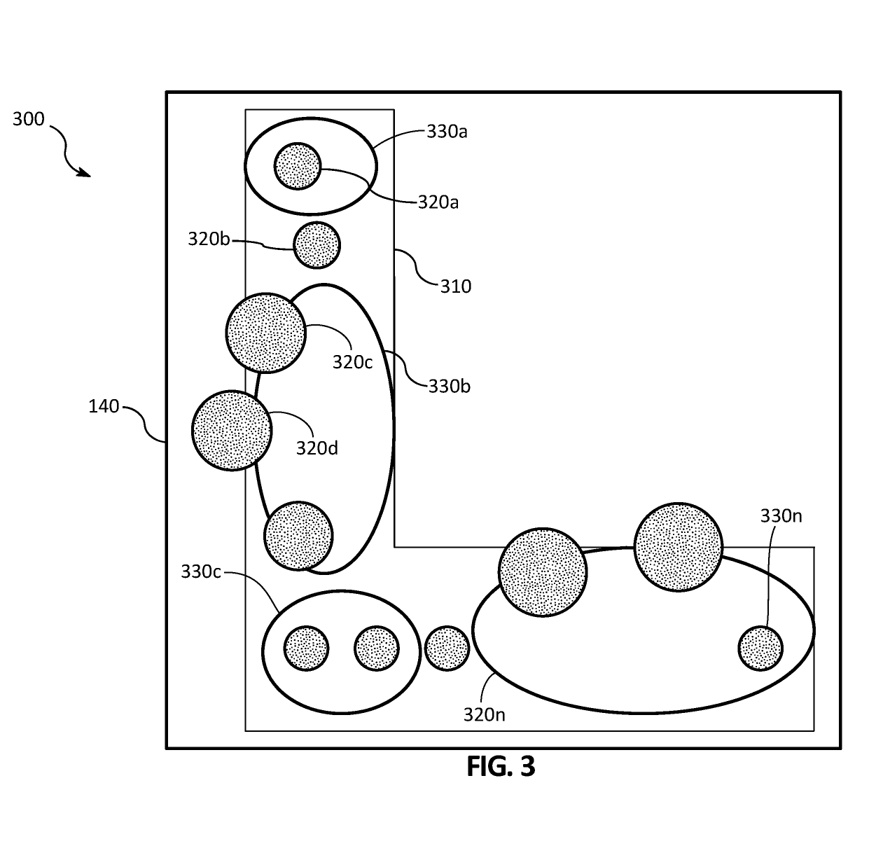

For example, FIG. 3 depicts an example visual feedback configuration 300 that is translating the motion-based behavior skill of velocity into visual feedback. The magnitude of the velocity is represented by a radius of a circle. Visual feedback configuration 300 includes user interface 140 that displays a previously generated pattern 310. User interface 140 also displays previously generated motion-based behavior data 330(a-n), where n is an integer greater than or equal to one, generated from the creation of previously generated pattern 310 and motion-based behavior data 320(a-n), where n is an integer greater than or equal to one, generated from a trace of previously generated pattern 310.

In this example, previously generated pattern 310 is a pattern of an “L”. However, as noted above, previously generated pattern 310 may be any pattern generated by the individual, another individual, and/or computing device that also generates previously generated motion-based behavior data 330(a-n). In this example, a second individual different from the individual that has experienced a less of a decline in their movement control as compared to the individual completes previously generated pattern 310. The second individual generates previously generated motion-based behavior data 330(a-n) when completing previously generated pattern 310. In this example, previously generated motion-based behavior data 330(a-n) is the velocity in which the second individual completes previously generated pattern 310 at each point in previously generated pattern 310. As noted above, previously generated motion-based behavior data 330(a-n) may be any kind of motion-based behavior data generated from a motion-based behavior skill that when maintained at an adequate level may slow the rate in which the individual's movement control declines.

In this example, previously generated motion-based behavior data 330(a-n) is translated into visual feedback as represented by circles. The magnitude of the velocity captured at each point in generating previously generated pattern 310 by the second individual is represented by the radius of each circle in previously generated motion-based behavior data 330(a-n). As the second individual increases the velocity in generating previously generated pattern 310, the radius of each circle representing the velocity increases. As the second individual decreases the velocity in generating previously generated pattern 310, the radius of each circle representing the velocity decreases.

For example, the second individual initiates the generation of previously generated pattern 310 with the circle representing previously generated motion-based behavior data 330a. The circle 330a has a smaller radius as compared to the other circles 330(b-n) representing the second individual beginning previously generated pattern 310 with a lower velocity. The circles representing previously generated motion-based behavior data 330b has a larger radius as compared to circle 330a indicating that the second individual increased the velocity during that portion of the previously generated pattern 310. Circles 330(a-n) also are within the outline of previously generated pattern 310 so that the second individual not only completed previously generated pattern 310 with a higher velocity but was also precise indicating that the second individual has had a minimal degradation in their movement control.

After the second individual completes previously generated pattern 310, the individual then completes the trace of previously generated pattern 310. The individual initiates the trace of previously generated pattern 310 with the circle representing motion-based behavior data 320a. The circle has a significantly smaller radius than any of the circles 330(a-n) generated by the second individual indicating that the individual begins the trace of previously generated pattern 310 with a significantly lower velocity. The circles representing motion-based behavior data 320c and 320d have a larger radii compared to circles 320a and 320b generated by the individual but significantly smaller radii than circles 330(a-n) generated by the second individual. This indicates that the individual increased the velocity in regards to circles 320a and 320b relative to circle 320a but did not reach the velocity in which the second individual generated previously generated pattern 310.

Further, circles 320c and 320d with larger radii indicating that the individual increases the velocity fall outside of the outline of previously generated pattern 310 are also less precise due to the individual attempting to increase the velocity. Circles 330(a-n) generated by the second individual not only provide visual feedback of higher velocities than circles 320(a-n) generated by the individual but also greater precision in that each circle 330(a-n) falls within the outline of previously generated pattern 310 while circles 320c and 320d fall outside the outline of previously generated pattern 310.

The visual feedback generated from the velocity of the trace of previously generated pattern 310 as depicted by circles 330(a-n) may be easily comprehended by the individual so that the individual may make the efforts to adjust the velocity when necessary. The translation of the magnitude of the velocity to the radius of each circle 330(a-n) may enable the individual to comprehend when the individual is generating slower velocities as compared to generating faster velocities in completing the trace. The individual may easily increase the velocity in which they are completing the trace when each circle 330(a-n) depict shorter radii. Each time the individual completes the trace, the individual may concentrate on increasing the velocity in completing the trace while attempting to maintain the precision in completing the trace within the outline of previously generated pattern 310. Thus, the individual may continue to sharpen the necessary skills to decrease the rate in which the individual's movement control declines.

The visual feedback generated from the velocity of the trace may be calculated based on the location of where the individual is engaging the multi-touch screen of user interface 140. At each point the individual engages the multi-touch screen of user interface 140, the x-coordinate and the y-coordinate relative to the multi-touch screen as well as a time stamp for each point may be obtained. A clock may be initiated when the individual first engages the multi-touch screen at the initial point of previously generated pattern 310.

As the individual continues the trace, the x-coordinate and the y-coordinate relative to the multi-touch screen may be obtained as well as the time provided by the clock of when the individual has reached each respective x-coordinate and y-coordinate. The distance travelled between each x-coordinate and y-coordinate may then be calculated. The velocity at each x-coordinate and y-coordinate may then be determined based on the distance travelled from the previous x-coordinate and y-coordinate and the time provided by the clock at which the individual reached each respective x-coordinate and y-coordinate. The radius of each respective circle 330(a-n) may then be generated based on the velocity.

The visual feedback may not be limited to a circle where the radius of the circle is adjusted based on the velocity. The visual feedback may include but is not limited to adjusting the color of the trace, adjusting the thickness of the line of the trace, and/or any other type of visual feedback where the intensity of the visual feedback is adjusted to represent a change in magnitude of the motion-based behavior data represented by the visual feedback that will be apparent to those skilled in the relevant art(s) without departing from the spirit and scope of the present disclosure. In an example embodiment, step 250 may be performed by generating module 470 as shown in FIG. 4 and discussed in more detail below.

As noted above, the individual may experience a further impact in slowing the rate of decline in their movement control when the level of each motion-based behavior skill that the individual is to strive for when completing the trace of the pattern is determined by another individual. If the individual has experienced a significant decline in their movement control, the individual may no longer have the aptitude to independently grasp the level that each motion-based behavior skill is to reach when completing the trace of the pattern to adequately slow the rate of decline in their movement control. For example, the individual may complete the trace at a specific velocity. Despite the visual feedback providing visible comprehension to the individual in regards to the level of velocity, the individual may still not comprehend that the individual should be striving to complete the trace at higher velocities in order to slow the rate of decline in their movement control.

Thus, the individual may require that a benchmark associated with motion-based behavior skills be put in place for the individual to strive for when completing the trace. Not only does the individual strive for precision in tracing previously generated trace 310 with x-coordinates and y-coordinates within a threshold of previously generated trace 310, the individual may also strive for other motion-based behavior skills, such as velocity, to be within a threshold of those implemented by the second individual. The benchmark may be at a level that when reached consistently by the individual may slow the rate of decline of the individual's movement control. The individual may then strive to reach the benchmark with the motion-based behavior skills each time the individual completes the trace.

For example, a second individual that has experienced minimal decline in their movement control, such as a physical therapist or a younger family member, may first complete previously generated pattern 310 with motion-based behavior skills at higher levels than the individual that is suffering a decline in their movement control. The individual may then attempt to trace the pattern completed by the second individual and in doing so strive to have each motion-based behavior skill reach the level that the second individual reached for each respective motion-based behavior skill.

In step 260, the motion-based behavior data may be compared with previously captured motion-based behavior data. The comparison in the motion-based behavior data and the previously captured motion-based behavior data may be executed to determine whether the individual when completing the trace generated the motion-based behavior data is within a threshold of the previously captured motion-based behavior data. Such a comparison may determine whether the individual generated motion-based behavior data that is sufficient to slow the rate of decline of the individual's movement control as will be discussed in greater detail below. The motion-based behavior data associated with the individual and the previously captured motion-based behavior data may be stored in motion-based behavior data database 190.

Each time the individual engages user interface 140 to complete a trace, the motion-based behavior data generated by each trace may be stored in motion-based behavior data database 190 as associated with the individual. As a result, motion-based behavior data database 190 continues to accumulate motion-based behavior data associated with the individual each time the individual engages in the authentication session and traces the pattern. The motion-based behavior data generated from the present trace of the pattern for the present authentication session may be compared to the previously captured motion-based behavior data accumulated in the motion-based behavior data database 190. In an example embodiment, step 260 may be performed by comparing module 480 as shown in FIG. 4 and discussed in more detail below.

In an embodiment, the comparison of the motion-based behavior data generated by the individual to the previously generated motion-based behavior data generated by the second individual may be the basis of a competition between the individual and the second individual. Generating a competition from the comparison of the motion-based behavior data to the previously generated motion-based behavior data may provide additional incentive for the individual to strive to have their motion-based behavior skills reach the benchmark necessary to slow the rate of decline of the individual's movement control.

However, the individual may rarely if ever generate a trace with motion-based behavior skills similar to that of the second individual when a significant disparity exists in the motion-based behavior skills between the individual and the second individual. The individual may become discouraged in engaging in the competition and decide to no longer engage in the tracing of previously generated pattern 310 when the individual consistently fails to generate motion-based behavior data similar to the previously generated motion-based behavior data. Thus, a threshold between the motion-based behavior data and the previously generated data that provides an adequate benchmark for the individual to strive for while being realistic as well so that the individual successfully reach the benchmark and be encouraged to continue with the competition.

Step 270 is performed when the threshold between the motion-based behavior data and the previously captured motion-based behavior data is determined. The threshold may be determined based on the skill level of the motion-based behavior skills of the individual as compared to the skill level of the motion-based behavior skills of the second individual. For example, the threshold may be greater when the disparity in the skill levels of the motion-based behavior skills between the individual and the second individual are greater. In another example, the threshold may be lesser when the disparity in the skill levels of the motion-based behavior skill between the individual and the second individual are lesser.

In an embodiment, the threshold may be manually selected. In such an embodiment, the second individual may be someone, such as a physical therapist, with an expertise in the current skill level of the individual and the benchmark that the individual is to strive for to slow the decline in the individual's movement control. As a result, the second individual may intelligently select the threshold so that the individual strives for a benchmark that continues the progress of the individual in sharpening their motion-based behavior skills while yet ensuring a moderate success rate so that the individual continues to engage the competition.

For example, the physical therapist may manually select the threshold on visual feedback multi-touch device 110 based on the individual's current progress in their motion-based behavior skills as evaluated by the physical therapist in their current session. The physical therapist may generate previously generated pattern 310 at a velocity and with a precision well beyond the current capabilities of the individual. However, the physical therapist may select the threshold so that the individual strives for a benchmark that slows the decline of their movement control while allowing the individual to successfully come within the selected threshold of the physical therapist's velocity and precision 40% of the time. Such a threshold is sufficient so that the individual continues to progress in slowing the decline of their movement control while allowing the individual to experience sufficient success so that the individual does not become discouraged.

In an embodiment, the threshold may be automatically selected. In such an embodiment, the individual may have suffered a significant decline in their movement control as compared to the second individual in which an increased threshold between the individual and the second individual may be automatically selected. Also in this embodiment, the individual and the second individual may have suffered similar declines in their movement control so that a decreased threshold between each may be automatically selected.

The threshold may be selected based on input provided by the individual and the second individual. For example, the individual may provide their age and also whether they are engaging in the competition to improve their movement control. The second individual may provide their age and whether they are engaging in the competition to improve their movement control.

In such an example, the individual inputs an age that is greater than 70 as well as confirming they are engaging the competition to improve their movement control and the second individual inputs an age that less than 30 and is not engaging in the competition to improve their movement control. An increased threshold is then automatically selected based on the assumption that the precision and the velocity generated by the individual will be significantly weaker than the precision and the velocity generated by the second individual. As a result, an increased threshold is necessary so that the individual experiences some success in attempting to replicate the precision and the velocity of the second individual.

In another example, both the individual and the second individual enter ages that are greater than 70 and are both engaging the competition to improve their movement control. A decreased threshold is then automatically selected based on the assumption that the precision and the velocity generated by the individual and the second individual may be similar. As a result, the individual and the second individual may compete on the merits to replicate the precision and the velocity of each other without a threshold.

The threshold may also be automatically selected based on motion-based behavior data previously generated by the individual during previous traces as well as previously generated motion-based behavior data generated by the second individual during previous generations of previously generated pattern 310. Each time that the individual and the second individual engage in completing the trace and/or pattern, the individual and the second individual may log-in so that each may be identified. After the second individual has logged in, the previously generated motion-based behavior data captured from the generation of each previously generated pattern 310 may be stored in motion-based behavior data database 190 as associated with the second individual. After the individual has logged in, the motion-based behavior data captured from the generation of each trace of previously generated pattern 310 may be stored in motion-based behavior data database 190 as associated with the individual.

For each subsequent log-in for the second individual, the previously generated motion-based behavior data associated with the second individual may be retrieved from motion-based behavior data database 190. The retrieved previously generated motion-based behavior data may be evaluated to determine whether significant variance existed in the previously generated motion-based behavior data for each generation of previously generated pattern 310. A significant variance in each previously generated pattern 310 may indicate that the second individual may have suffered a decline in their movement control and may be classified as such. Slight variance in each previously generated pattern 310 may indicate that the second individual has not suffered any decline in their movement control and may be classified as such.

The motion-based behavior data associated with the individual may be retrieved from motion-based behavior data database 190 and analyzed in a similar fashion as discussed above regarding the second individual. The variance in the motion-based behavior data associated with the individual may then be compared with the variance in the previously generated motion-based behavior data associated with the second individual. Based on this comparison, the threshold may be automatically selected.

For example, an increased threshold is automatically selected when the variance associated with the individual is significant and the variance associated with the second individual is much less significant. Such a difference in variance indicates that the precision and velocity generated by the individual will be significantly weaker than the precision and the velocity generated by the second individual. As a result, an increased threshold is necessary so that the individual experiences some success in attempting to replicate the precision and the velocity of the second individual.

In another example, a decreased threshold is automatically selected when the variance associated with the individual is slight and the variance associated with the second individual is slight. Slight differences in variance indicate that the precision and the velocity generated by the individual and the second individual may be similar. As a result, the individual and the second individual may compete on the merits to replicate the precision and the velocity of each other without a threshold. In an example embodiment, step 270 may be performed by determination module 460 as shown in FIG. 4 and discussed in more detail below.

After step 270 is completed, the trace generated by the individual of previously generated pattern 310 may be authenticated as a match or rejected as a match. Step 280 is performed when the trace is authenticated as a match. The trace may be authenticated as a match when the motion-based behavior data is within the threshold of the previously captured motion-based behavior data as determined in step 270. In an example embodiment, step 280 may be performed by authentication module 430 as shown in FIG. 4 and discussed in more detail below.

Step 290 is performed when the trace is rejected as a match. The trace is rejected as a match when the motion-based behavior data is outside the threshold of the previously captured motion-based behavior data as determined in step 270. In an example embodiment, step 290 may be performed by rejection module 450 as shown in FIG. 4 and discussed in more detail below.

In an embodiment, the above steps may be implemented in a game between the individual and the second individual. The second individual may first generate previously generated pattern 310 where user interface 140 displays previously generated pattern 310 with visual feedback. The individual may then trace previously generated pattern 310 in an attempt to match the precision and the velocity generated by the individual to the precision and the velocity generated by the second individual in generating previously generated pattern 310.

The individual may begin with a total score. For example, the individual starts with a total score of 100. After the individual completes the trace of previously generated pattern 310, an error score may be assessed to the trace. The error score is based on how much the precision and the velocity generated from the trace differentiate from the threshold determined between the individual and the second individual. The error score may increase as the difference between the precision and the velocity and threshold increases. The error score may decrease as the difference between the precision and the velocity and the threshold decreases. For example, the individual completed the trace with precision and velocity that are significantly different from the threshold. As a result, an error score of 30 is assessed to the trace. The error score assessed to the trace completed by the individual may then be deducted from the individual's total score. For example, the error score of 30 assessed to the trace completed by the individual may be deducted from the individual's total score of 100 giving the individual a current score of 70.

The individual may then generate previously generated pattern 310 that is displayed by user interface 140 with visual feedback. The second individual may trace previously generated pattern 310 in an attempt to match the precision and the velocity generated by the second individual to the precision and the velocity generated by the individual. An error score may then be assessed to the second individual's trace and deducted from the second individual's total score. For example, the second individual has a total score of 100. The second individual completed the trace with precision and velocity that are similar to the precision and velocity generated by the individual. As a result, an error score of 5 is assessed to the trace by the second individual and is deducted from the second individual's total score of 100 giving the second individual a current score of 95. The above process may be repeated until either the individual or the second individual has enough error deductions so that their current score reaches 0. At the point, the person that reaches 0 first loses the game.

The above game may provide an unending source of novelty that contributes to slowing the decline of the individual's movement control. Rather than tracing the same pattern over and over again, the above game provides a new pattern to trace with different precision and velocities to match each time the second individual generates a new previously generated pattern 310. Such randomness in the generation of patterns not only prevents the individual from becoming bored that may deter the individual from engaging in the game but also contributes to slowing the decline of the individual's movement control.

The above game also forces the individual to implement movement planning before and/or during the execution of the trace which also contributes to slowing the decline of the individual's movement control. Before the individual begins the trace, the individual must first memorize the sequence that the second individual completed previously generated trace 310 with and then begin the trace with the same initial point that the second individual. The individual must then follow the appropriate sequence while attempting to match the precision and the velocity of the second individual. The individual when completing previously generated trace 310 also implements movement planning because the individual must first think about the pattern that they are going to generate and then generate that pattern. Such movement planning contributes to slowing the decline of the individual's movement control.

The interaction between the individual and the second individual may not be limited to a single visual feedback multi-touch device 110 in that both the individual and the second individual share a single visual feedback multi-touch device 110. Rather, the individual may use a first visual feedback multi-touch device 110 and the second individual may use a second visual feedback multi-touch device. The second individual may generate previously generated pattern 310 on second visual feedback multi-touch device. Previously generated pattern 310 may then be transmitted via network 120 to visual feedback multi-touch device 110 so that the individual may then trace previously generated pattern 310 via visual feedback multi-touch device 110. The trace of previously generated pattern 310 may then be transmitted via network 120 to second visual feedback multi-touch device for the second individual to examine. The generation of previously generated pattern 310 and the trace may be streamed in real-time via network 120 to visual feedback multi-touch device 110 and second visual feedback multi-touch device so that the individual and the second individual may observe the generation of each in real-time.

### Example Visual Feedback System

As shown in FIG. 4, motion-based identity authentication system 400 includes motion-based sensor server 150, network 120, motion-based sensor system 130, visual feedback multi-touch device 110, user interface 140, and motion-based behavior data database 190. Visual feedback multi-touch device 110 includes a generating module 470, a transceiver 420, a capturing module 440, a comparing module 480, an authentication module 430, a rejection module 450, and a determination module 460.

Modules as described above may be used by visual feedback multi-touch device 110. Examples of functionality performed by each module are referenced in the above discussion. However, the above references are examples and are not limiting. The functionality of each module may be performed individually by each module and/or be shared among any combination of modules. As referred to herein, a module may be any type of processing (or computing) device having one or more processors. For example, a module can be an individual processor, workstation, mobile device, computer, cluster of computers, set-top box, game console or other device having at least one processor. In an embodiment, multiple modules may be implemented on the same processing device. Such a processing device may include software, firmware, hardware, or a combination thereof. Software may include one or more applications and an operating system. Hardware can include, but may not be limited to, a processor, memory, and/or graphical user display.

Embodiments can work with software, hardware, and/or operating system implementations other than those described herein. Any software, hardware, and operating system implementations suitable for performing the functions described herein can be used. Embodiments are applicable to both a client and to a server or a combination of both.

The breadth and scope of the present disclosure should not be limited by any of the above-described example embodiments, but should be defined only in accordance with the following claims and their equivalents.

What is claimed is:

1. A multi-touch device for generating visual feedback from a traced pattern that is completed by an individual, comprising:
a user interface configured to display a previously generated pattern by a user interface of the multi-touch device to the individual for the individual to trace;
a transceiver configured to receive the traced pattern generated from continuously tracing the previously generated pattern by the individual from an initial point on the previously generated pattern to an end point on the previously generated pattern via the user interface; and
a generating module configured to generate visual feedback via the user interface that translates motion-based behavior data associated with the traced pattern to a visible depiction of the motion-based behavior data.

2. The multi-touch device of claim 1, wherein the transceiver is further configured to receive the previously generated pattern before the traced pattern that is continuously generated with the previously captured motion-based behavior data from an initial point on the user interface to an end point on the user interface.

3. The multi-touch device of claim 2, wherein the generating module is further configured to:
adjust an intensity of the visual feedback as a magnitude associated with the motion-based behavior data that is depicted by the visual feedback increases or decreases; and
adjust an intensity of the visual feedback as a magnitude associated with the previously captured motion-based behavior data that is depicted by the visual feedback increases or decreases.

4. The multi-touch device of claim 3, wherein the generating module is further configured to:
increase the intensity of the visual feedback as a magnitude associated with a velocity that the individual completes the traced pattern with increases; and
decrease the intensity of visual feedback as the magnitude associated with the velocity that the individual completes the traced pattern with decreases.

5. The multi-touch device of claim 1, further comprising:
a comparing module configured to compare motion-based behavior data associated with the traced pattern with previously captured motion-based behavior data associated with the previously generated pattern; and

[symbol]

6. The multi-touch device of claim 5, further comprising:
an evaluation module configured to:
evaluate whether the motion-based behavior data is within a threshold of the previously captured motion-based behavior data;
identify the traced pattern as a match to the previously generated pattern when the motion-based behavior data is within the threshold of the previously captured motion-based behavior data; and
reject the traced pattern as not a match to the previously generated pattern when the motion-based behavior data is outside the threshold of the previously captured motion-based behavior data.

7. The multi-touch device of claim 6, wherein the threshold is manually selected.

8. The multi-touch device of claim 6, further comprising:
a selection module configured to:
compare the motion-based behavior data generated by the individual for each previous instance completed by the traced pattern;
determine a level of variance in the motion-based behavior data generated by the individual for each previous instance that the individual completed the traced pattern; and
automatically select the threshold associated with the individual based on the level of variance in the motion-based behavior data generated by the individual for each previous instance that the individual completed the traced pattern.

9. The multi-touch device of claim 8, wherein the selection module is further configured to:
increase the threshold associated with the individual when the level of variance in the motion-based behavior data generated by the individual for each previous instance that the individual completed the trace increases; and
decrease the threshold associated with the individual when the level of variance in the motion-based behavior data generated by the individual for each previous instance that the individual completed the traced pattern decreases.

10. The multi-touch device of claim 9, wherein the storage module is further configured to store the motion-based behavior data generated by the individual for each previous instance that the individual completed the traced pattern in a motion-based behavior data database.

11. A method for generating visual feedback from a traced pattern that is completed by an individual using a multi-touch device, comprising:
displaying a previously generated pattern by a user interface of the multi-touch device to the individual for the individual to trace;
receiving the traced pattern generated from continuously tracing the previously generated pattern by the individual from an initial point on the previously generated pattern to an end point on the previously generated pattern via the user interface of the multi-touch device; and
generating visual feedback via the user interface that translates motion-based behavior data associated with the traced pattern to a visible depiction of the motion-based behavior data.

12. The method of claim 11, further comprising:
receiving the previously generated pattern before the traced pattern that is continuously generated with previously captured motion-based behavior data from an initial point on the user interface to an end point on the user interface.

13. The method of claim 12, wherein the generating comprises:
adjusting an intensity of the visual feedback as a magnitude associated with the motion-based behavior data that is depicted by the visual feedback increases or decreases; and
adjusting an intensity of the visual feedback as a magnitude associated with the previously captured motion-based behavior data that is depicted by the visual feedback increases or decreases.

14. The method of claim 13, wherein the generating further comprises:
increasing the intensity of the visual feedback as a magnitude associated with a velocity that the individual completes the traced pattern with increases; and
decreasing the intensity of the visual feedback as the magnitude associated with the velocity that the individual completes the traced pattern with decreases.

15. The method of claim 11, further comprising:
comparing the motion-based behavior data associated with the traced pattern with the previously captured motion-based behavior data associated with the previously generated pattern.

16. The method of claim 15, further comprising:
evaluating whether the motion-based behavior data is within a threshold of the previously captured motion-based behavior data;
identifying the traced pattern as a match to the previously generated pattern when the motion-based behavior data is within the threshold of the previously captured motion-based behavior data; and
rejecting the traced pattern as not a match to the previously generated pattern when the motion-based behavior data is outside the threshold of the previously captured motion-based behavior data.

17. The method of claim 16, wherein the threshold is manually selected.

18. The method of claim 16, further comprising:
comparing the motion-based behavior data generated by the individual for each previous instance that the individual completed the traced pattern;
determining a level of variance in the motion-based behavior data generated by the individual for each previous instance that the individual completed the traced pattern; and
automatically selecting the threshold associated with the individual based on the level of variance in the motion-based behavior data generated by the individual for each previous instance that the individual completed the traced pattern.

19. The method of claim 18, further comprising:
increasing the threshold associated with the individual when the level of variance in the motion-based behavior data generated by the individual for each previous instance that the individual completed the traced pattern increases; and
decreasing the threshold associated with the individual when the level of variance in the motion-based behavior data generated by the individual for each previous instance that the individual completed the traced pattern decreases.

20. The method of claim 18, further comprising:
storing the motion-based behavior data generated by the individual for each previous instance that the individual completed the traced pattern in a motion-based behavior data database.
